(19) United States
(12) Patent Application Publication
(10) Pub. No.: US 2025/0169871 A1
(43) Pub. Date: May 29, 2025

(54) EMITTER FOR INTRAVASCULAR LITHOTRIPSY DEVICE WITH COATING FOR ENHANCED DURABILITY

(71) Applicant: BOLT MEDICAL, INC., Carlsbad, CA (US)

(72) Inventor: Eric Schultheis, San Clemente, CA (US)

(21) Appl. No.: 18/902,649

(22) Filed: Sep. 30, 2024

Related U.S. Application Data
(60) Provisional application No. 63/604,001, filed on Nov. 29, 2023.

(51) Int. Cl. A61B 18/04; A61B 18/00; A61B 18/26

(52) U.S. Cl. CPC A61B 18/042; A61B 2018/00148; A61B 2018/0022; A61B 2018/00345; A61B 18/26

(57) ABSTRACT

A catheter system (200) for treating a treatment site (106) includes an energy source (124), an energy guide (222A) and an emitter (231). The energy guide (222A) receives energy (243) from the energy source (124). The emitter (231) includes (i) a guide distal end (222D) of the energy guide (222A), (ii) a plasma target (233) spaced apart from the guide distal end (222D), the energy guide (222A) emitting the energy (243) away from the guide distal end (222D) and toward the plasma target (233) so that plasma (134) is generated at the plasma target (233), and (iii) an emitter housing (260) secured to the energy guide (222A) and one of secured to and integrally formed with the plasma target (233) to maintain a relative position between the guide distal end (222D) and the plasma target (233). The emitter housing (260) includes a housing surface (260S), with a coating (260C) being provided on the housing surface (260S). The housing surface (260S) is formed from a first material, and the coating (260C) is formed from a second material that is different than the first material.

### RELATED APPLICATION

This Application claims priority on U.S. Provisional Patent Application Ser. No. 63/604,001 filed on Nov. 29, 2023, and entitled “EMITTER FOR INTRAVASCULAR LITHOTRIPSY DEVICE WITH COATING FOR ENHANCED DURABILITY”. To the extent permitted, the contents of U.S. Application Ser. No. 63/604,001 are incorporated in their entirety herein by reference.

### BACKGROUND

Vascular lesions at a treatment site within and/or adjacent to vessels in the body can be associated with an increased risk for major adverse events, such as myocardial infarction, embolism, deep vein thrombosis, stroke, and the like. Severe vascular lesions, such as severely calcified vascular lesions, can be difficult to treat and achieve patency for a physician in a clinical setting.

Vascular lesions may be treated using interventions such as drug therapy, balloon angioplasty, atherectomy, stent placement, and vascular graft bypass, to name a few. Such interventions may not always be ideal or may require subsequent treatment to address the lesion.

Intravascular lithotripsy is one method that has been recently used with some success for breaking up vascular lesions within vessels in the body. Intravascular lithotripsy utilizes a combination of pressure waves and bubble dynamics that are generated intravascularly in a fluid-filled balloon catheter. In particular, in certain implementations of an intravascular lithotripsy treatment, a high energy source is used to provide energy to an energy guide and/or an emitter in order to generate plasma and ultimately pressure waves as well as a rapid bubble expansion within a fluid-filled balloon to crack calcification at a treatment site within the vasculature that includes one or more vascular lesions. The associated rapid bubble formation from the plasma initiation and resulting localized fluid velocity within the balloon transfers mechanical energy through the incompressible fluid to impart a fracture force on the intravascular calcium, which is opposed to the balloon wall. The rapid change in fluid momentum upon hitting the balloon wall is known as hydraulic shock, or water hammer.

Generation of a plasma within an intravascular lithotripsy catheter typically requires a significant amount of energy in a short amount of time, during which the energy is converted into a therapeutic bubble and/or a therapeutic pressure wave. With sufficiently high energy and short pulse durations, the generation of the plasma near a distal end of a small diameter energy guide and/or an emitter housing, which can form at least a portion of the emitter, creates a potential for damage to and/or imperfections on the distal end of the energy guide and/or the emitter housing of the emitter due to various factors. Such factors include, but are not limited to, its proximity to the plasma generation and/or the pressure wave, high plasma temperatures, and waterjet from collapse of the bubble, as non-exclusive examples.

As such, during creation of the desired plasma, the lithotripsy emitters are subjected to high localized acoustic pressures that are generated from the plasma formation during therapy. In various implementations, the lithotripsy emitters, including the emitter housing, can be extremely small in size in order to keep the crossing profile of the catheter as small as possible, which is a desirable feature for the catheter. At these small sizes, damage to and/or imperfections on the emitters and/or the emitter housing such as crevices, rough surfaces, microcracks, surface irregularities, and the like can cause the emitters and/or the emitter housing to fracture during use when acoustic energy is created. Thus, it is desired to provide the emitters and/or the emitter housing with a means of protection in order to provide enhanced durability for the emitters.

### SUMMARY

The present invention is directed toward a catheter system for placement within a blood vessel having a vessel wall or within a heart valve within a body of a patient. The catheter system can be used for treating a treatment site within or adjacent to the vessel wall of the blood vessel, or within or adjacent to the heart valve, within the body of the patient. In various embodiments, the catheter system includes an energy source, an energy guide, and an emitter. The energy source generates energy. The energy guide receives the energy from the energy source. The energy guide has a guide distal end. The emitter includes (i) the guide distal end of the energy guide, (ii) a corresponding plasma target that is spaced apart from the guide distal end, the energy guide emitting the energy in a direction away from the guide distal end and toward the plasma target so that a plasma is generated at the plasma target upon receiving the energy from the energy guide, and (iii) an emitter housing that is secured to the energy guide and is one of secured to and integrally formed with the plasma target so as to maintain a relative position between the guide distal end of the energy guide and the plasma target. The emitter housing includes a housing surface, and with a coating being provided on the housing surface. In many embodiments, the housing surface is formed at least in part from a first material; and the coating is formed at least in part from a second material that is different than the first material.

In some embodiments, the housing surface is formed at least in part from one or more of titanium, stainless steel, tungsten, tantalum, platinum, molybdenum, niobium, and iridium.

In certain embodiments, the housing surface is formed at least in part from tungsten.

In some embodiments, the housing surface is formed at least in part from one or more of a polymer, a polymeric material, a plastic, and nylon.

In certain embodiments, the plasma target is formed from the same materials as the housing surface.

In various embodiments, the plasma target is formed from different materials than the housing surface.

In some embodiments, the coating that is provided on the housing surface is formed at least in part from one or more of CVD diamond, diamond, an adhesive, a spray-coated adhesive, and a gold plating.

In certain embodiments, the coating that is provided on the housing surface is formed at least in part from CVD diamond.

In some embodiments, the coating that is provided on the housing surface has a thickness of between approximately one micron and fifteen millimeters.

In various embodiments, the coating that is provided on the housing surface has a thickness of between approximately three microns and eight millimeters.

In certain embodiments, the emitter housing includes (i) a first housing section that is secured to the energy guide at or near the guide distal end, (ii) a second housing section that is one of secured to and integrally formed with the plasma target, and (iii) a connector section that is coupled to and extends between the first housing section and the second housing section.

In some embodiments, the first housing section includes a guide aperture, at least a portion of the energy guide being secured within the guide aperture. In certain embodiments, the catheter system further includes adhesive material that secures the first housing section to the energy guide at or near the guide distal end.

In various embodiments, the second housing section includes a target aperture, at least a portion of the plasma target being secured within the target aperture. In certain embodiments, the catheter system further includes adhesive material that secures the second housing section to the plasma target.

In certain embodiments, the second housing section is integrally formed with the plasma target.

In some embodiments, the plasma generated at the plasma target is in the form of a plasma bubble; and the connector section includes a section opening, the plasma target being configured to direct the energy from the plasma bubble through the section opening and toward the treatment site.

In certain embodiments, the plasma target has a proximal end that is angled so the energy from the plasma bubble is directed through the section opening and toward the treatment site.

In various embodiments, the plasma target is spaced apart from the guide distal end by a target gap distance that is greater than 1 μm.

In some embodiments, the catheter system further includes an inflatable balloon that encircles the guide distal end of the energy guide, the plasma target being positioned within the inflatable balloon.

In certain embodiments, the energy guide includes a distal region having a longitudinal axis, and the direction the energy is emitted from the energy guide and toward the plasma target is substantially along the longitudinal axis of the distal region.

In many embodiments, the energy source is a laser and the energy guide is an optical fiber.

In various embodiments, the present invention can also be directed toward a method for treating the treatment site, the method including the step of providing any one of the catheter systems shown and/or described herein.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic cross-sectional view illustration of an embodiment of a catheter system in accordance with various embodiments, the catheter system including an emitter assembly that includes at least one emitter having features of the present invention;

FIG. 2 is a simplified schematic side view illustration of a portion of an embodiment of the catheter system illustrated in FIG. 1, including an embodiment of the emitter having an emitter housing;

FIG. 3 is a simplified schematic perspective view illustration of a portion of another embodiment of the catheter system illustrated in FIG. 1, including another embodiment of the emitter and the emitter housing;

FIG. 4A is a simplified schematic side view illustration of a portion of an embodiment of the emitter, including an emitter housing without a coating provided on a housing surface of the emitter housing; and

FIG. 4B is a simplified schematic side view illustration of a portion of another embodiment of the emitter, including the emitter housing of FIG. 4A having a coating provided on the housing surface of the emitter housing.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions at treatment sites within a body of a patient can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a balloon catheter configured to advance to a vascular lesion, such as a calcified vascular lesion or a fibrous vascular lesion, at a treatment site located within a body of a patient. In certain implementations, the “treatment site” can be located at or near a vessel wall of a blood vessel of the patient. Additionally, or in the alternative, in other implementations, the “treatment site” can be at or near a heart valve of the patient. Further, or in the alternative, in still other implementations, the “treatment site” can be at another suitable location within the body of the patient.

As defined herein, the terms “treatment site”, “intravascular lesion” and “vascular lesion” may be used interchangeably unless otherwise noted. The intravascular lesions and/or the vascular lesions are sometimes referred to herein as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is recognized that such a development effort might be complex and time-consuming, but would nevertheless be a routine undertaking of engineering for those of ordinary skill in the art having the benefit of this disclosure.

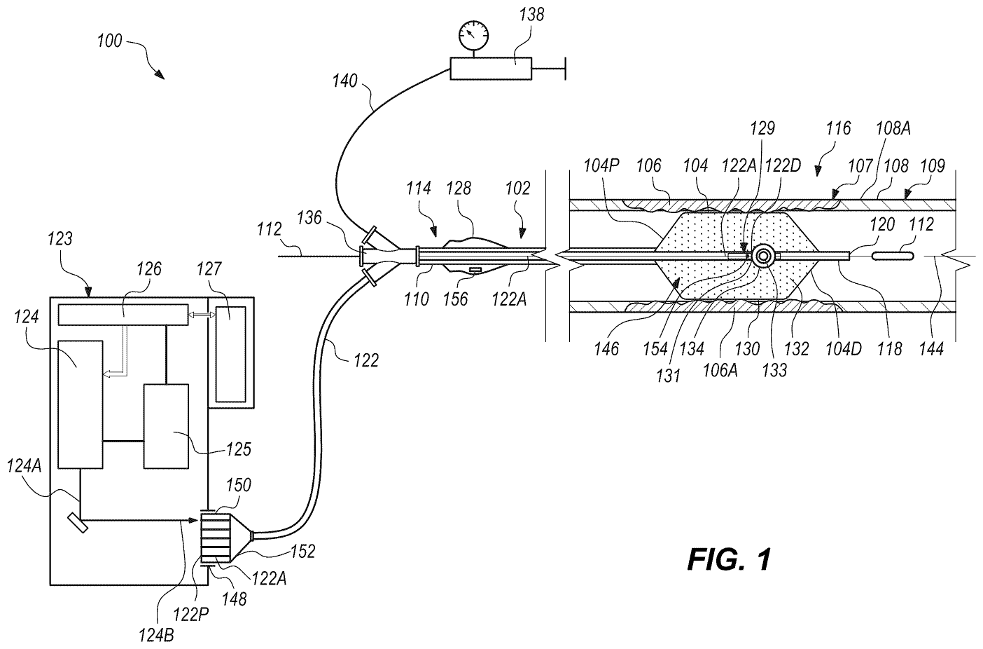

The catheter systems disclosed herein can include many different forms. Referring now to FIG. 1, a simplified schematic cross-sectional view illustration is shown of a catheter system 100 in accordance with various embodiments. The catheter system 100 is suitable for imparting pressure waves to induce fractures at one or more treatment sites 106 within or adjacent to a vessel wall 108A of a blood vessel 108, or on or adjacent to a heart valve, within a body 107 of a patient 109. In the embodiment illustrated in FIG. 1, the catheter system 100 can include (i) a catheter 102 including one or more of an inflatable balloon 104 (sometimes referred to herein simply as a “balloon”), a catheter shaft 110, a guidewire 112, a guidewire lumen 118, an energy guide bundle 122 including one or more energy guides 122A, a source manifold 136, a fluid pump 138, a handle assembly 128, and an emitter assembly 129; and (ii) a system console 123 including one or more of an energy source 124, a power source 125, a system controller 126, and a graphic user interface 127 (a “GUI”). In various embodiments, the emitter assembly 129 includes and/or incorporates at least one emitter 131 that is configured to direct and/or concentrate energy toward one or more vascular lesions 106A at the treatment site 106 within or adjacent to the vessel wall 108A of the blood vessel 108, or within or adjacent to a heart valve, within the body 107 of the patient 109. Alternatively, the catheter system 100, the catheter 102 and/or the system console 123 can include more components or fewer components than those specifically illustrated and described in relation to FIG. 1.

As illustrated in FIG. 1, the catheter 102 is configured to move to the treatment site 106 within or adjacent to the vessel wall 108A of the blood vessel 108 within the body 107 of the patient 109. The treatment site 106 can include one or more vascular lesions 106A such as calcified vascular lesions, for example. Additionally, or in the alternative, the treatment site 106 can include vascular lesions 106A such as fibrous vascular lesions. Still alternatively, in some implementations, the catheter 102 can be used at a treatment site 106 within or adjacent to a heart valve within the body 107 of the patient 109. Yet alternatively, in certain implementations, the catheter 102 can be used at a treatment site 106 at another suitable location within the body 107 of the patient 109.

The catheter shaft 110 can extend from a proximal portion 114 of the catheter system 100 to a distal portion 116 of the catheter system 100. The catheter shaft 110 can include a longitudinal axis 144. The guidewire lumen 118 is configured to move over the guidewire 112. As utilized herein, the guidewire lumen 118 defines a conduit through which the guidewire 112 extends. The catheter shaft 110 and/or the catheter 102 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106.

The balloon 104 can include a balloon proximal end 104P and a balloon distal end 104D. In certain embodiments, the balloon 104 can be coupled to the catheter shaft 110 and/or to the guidewire lumen 118. More particularly, in some embodiments, the balloon proximal end 104P can be coupled to the catheter shaft 110, and the balloon distal end 104D can be coupled to the guidewire lumen 118.

The balloon 104 includes a balloon wall 130 that defines a balloon interior 146. The balloon 104 can be selectively inflated with a catheter fluid 132 to expand from a deflated state suitable for advancing the catheter 102 through a patient's vasculature, to an inflated state (as shown in FIG. 1) suitable for anchoring the catheter 102 in position relative to the treatment site 106. Stated in another manner, when the balloon 104 is in the inflated state, the balloon wall 130 of the balloon 104 is configured to be positioned substantially adjacent to the treatment site 106.

As described, in various embodiments, the catheter system 100 and/or the emitter assembly 129 can include the at least one emitter 131 that is configured to transmit energy from the energy source 124 into the balloon interior 146 in order to generate plasma and/or pressure waves in the catheter fluid 132 within the balloon interior 146. Each of the emitters 131 includes a guide distal end 122D of one of the energy guides 122A, which is positioned within the balloon interior 146, and a corresponding plasma target 133 (also sometimes referred to as a “plasma generating structure” or a “plasma generator”) that is positioned near, but typically spaced apart from, the guide distal end 122D.

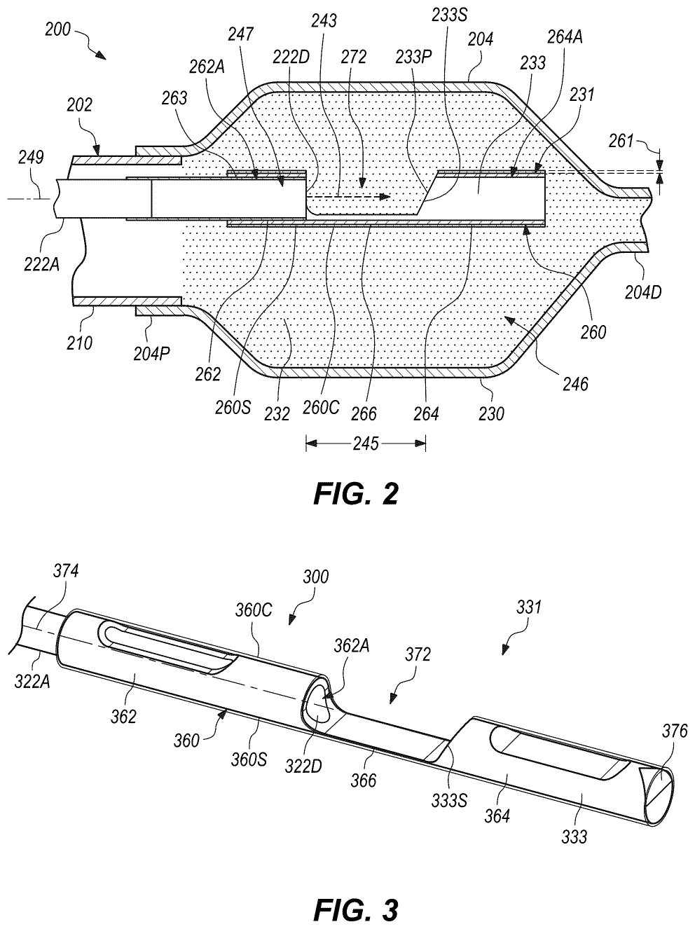

In various embodiments, each of the emitters 131 further includes an emitter housing 260 (illustrated in FIG. 2) that is configured to maintain a desired positioning between the guide distal end 122D of the energy guide 122A and the corresponding plasma target 133. As so defined, the emitters 131, and/or the components thereof, are configured to direct and/or concentrate energy generated in the catheter fluid 132 within the balloon interior 146 so as to impart pressure onto and induce fractures in the vascular lesions 106A at the treatment site 106.

More specifically, during use of the catheter system 100, energy from the energy source 124 is directed toward and received by the energy guide 122A, is guided through the energy guide 122A, and is then emitted from the guide distal end 122D of the energy guide 122A. The energy emitted from the guide distal end 122D is directed toward and contacts and energizes the corresponding plasma target 133 for purposes of generating the plasma in the catheter fluid 132 within the balloon interior 146.

As referred to herein, the plasma target 133 or “plasma generator” can include and/or incorporate any suitable type of structure that is located at or near the guide distal end 122D of the energy guide 122A. For example, in certain embodiments, the plasma target 133 can be provided in the form of a backstop-type structure with an angled face that redirects energy emitted from the guide distal end 122D toward the balloon wall 130 of the balloon 104 and/or toward the vessel wall 108A of the blood vessel 108 at the treatment site 106. Alternatively, the plasma target 133 can have another suitable structural design.

In many embodiments, the present invention utilizes a laser light source or other suitable light source as the energy source 124, and is configured to shine laser light energy onto the plasma target 133 to cause plasma generation via interaction with a plasma target material rather than optical breakdown of the catheter fluid 132. This moves the plasma creation away from the guide distal end 122D of the energy guide 122A (which can be an optical fiber in some embodiments). This can be accomplished by positioning the plasma target 133 away from the guide distal end 122D of the energy guide 122A to absorb the light energy and convert it into a plasma at some distance away from the guide distal end 122D of the energy guide 122A.

As an overview, in various embodiments, the emitter housing 260 can be thin, long and narrow, and can be subject to certain manufacturing irregularities that can weaken the overall structural integrity of the emitter housing 260. More specifically, during the manufacturing process and/or during repeated use of the catheter system 100 (and thus the emitter housing 260), the emitter housing 260 can include and/or develop small surface imperfections, such as cracks, crevices, surface roughness and/or other surface irregularities, that can adversely impact the structural strength of the emitter housing 260. In some embodiments, the emitter housing 260 can be formed from long, narrow tubing (a hypotube). Alternatively, in other embodiments, the emitter housing 260 can be fabricated by machining a single piece of rod material.

The emitter housing 260 can be formed from any suitable materials. For example, in certain non-exclusive embodiments, the emitter housing 260 can be formed from a hypotube including one or more metals such as titanium, stainless steel, tungsten, tantalum, platinum, molybdenum, niobium, iridium, etc. Alternatively, the emitter housing 260 may be formed from a hypotube including plastics such as polyimide and nylon. Still alternatively, the emitter housing 260 may be injection molded, and over molding processing can be used to secure the energy guide 122A and/or the plasma target 133 into place in a manner as described herein below. Yet alternatively, the emitter housing 260 can be formed in another suitable manner and/or from other suitable materials.

In many embodiments, the present invention further teaches coatings that may be applied to an intravascular lithotripsy emitter 131 to enhance the strength of the emitter 131. In particular, the emitter housing 260 can include a coating 260C (illustrated in FIG. 2) that is provided and/or applied on a housing surface 260S (illustrated in FIG. 2) of the emitter housing 260. The coating 260C can be formed from any suitable materials that can be used to provide enhanced protection and strength for the emitter housing 260, and, thus, enhanced durability for the emitter 131 as a whole. More specifically, the present invention incorporates the use of the coating 260C on the housing surface 260S of the emitter housing 260, with the coating 260C being formed, at least in part, from materials that are capable of filling in any cracks or crevices that may have formed on or into the housing surface 260S, smoothing out any rough surfaces within the housing surface 260S, and/or reducing a depth of any surface irregularities that may have been formed into the housing surface 260S. With such design, the unique coating 260C provided and/or applied on the housing surface 260S of the emitter housing 260 can help reduce any stress concentration areas inside of such cracks, crevices, surface roughness and/or surface irregularities.

For example, in certain embodiments, the coating 260C provided and/or applied on the housing surface 260S of the emitter housing 260 can include a chemical vapor deposition (CVD) diamond material. CVD diamond material is regularly used as a coating for tooling such as end mill and drill bits. This process is typically used for strengthening tungsten carbide tools to increase wear resistance. However, another advantage is the ability of the coating 260C to fill in the cracks and crevices as discussed above to reduce the depth of the cracks and crevices and therefore reduce the stress concentration zone at the bottom of the crack or crevice. Simply stated, the CVD diamond material coating can fill the cracks, crevices, and grain structure with CVD diamond material. The CVD diamond material is also one of the hardest known materials in existence, and can thus give extra strength to the housing surface 260S of the emitter housing 260.

An advantage of the CVD process is that a very finite and controllable thickness 261 (illustrated in FIG. 2) of the coating 260C can be applied to the component (the housing surface 260S of the emitter housing 260 in this particular implementation) from a few microns in thickness to several mm in thickness. Diamond material is a common material that is provided and/or applied using the CVD process and is also one of the hardest known materials in existence, thereby giving extra strength to the substrate.

However, it is appreciated that the coating 260C provided and/or applied on the housing surface 260S of the emitter housing 260 can alternatively be formed, at least in part, from any other suitable materials that can also achieve a hardened coating. For example, such alternative materials for the coating 260C include, but are not limited to, dip or spray-coated adhesives and/or polymers, gold plating, or any other suitable materials that can also be used to strengthen the housing surface 260S of the emitter housing 260 and/or the emitter 131 as a whole.

Thus, in many embodiments, as described, the housing surface 260S is formed, at least in part, from a first material, and the coating 260C is formed, at least in part, from a second material that is different than the first material. Alternatively, in other embodiments, the housing surface 260S and the coating 260C can be formed, at least in part, from one or more materials that are the same for both.

The balloon 104 suitable for use in the catheter system 100 includes those that can be passed through the vasculature of a patient 109 when in the deflated state. In some embodiments, the balloons 104 are made from silicone. In other embodiments, the balloon 104 can be made from materials such as polydimethylsiloxane (PDMS), polyurethane, polymers such as PEBAX™ material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In some embodiments, the balloon 104 can have a diameter (in the inflated state) ranging from at least two mm up to five mm.

In some embodiments, the balloon 104 can have a length ranging from at least three mm to 300 mm. More particularly, in some embodiments, the balloon 104 can have a length ranging from at least eight mm to 200 mm. It is appreciated that a balloon 104 having a relatively longer length can be positioned adjacent to larger treatment sites 106, and, thus, may be usable for imparting pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape.

In some embodiments, the balloon 104 can include a drug eluting coating or a drug eluting stent structure. The drug eluting coating or drug eluting stent can include one or more therapeutic agents including anti-inflammatory agents, anti-neoplastic agents, anti-angiogenic agents, and the like.

The catheter fluid 132 used to inflate the balloon 104 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the pressure waves are appropriately manipulated. In certain embodiments, the catheter fluid 132 suitable for use herein is biocompatible. A volume of catheter fluid 132 can be tailored by the chosen energy source 124 and the type of catheter fluid 132 used.

In certain embodiments, the catheter fluid 132 can include a wetting agent or surface-active agent (surfactant). These compounds can lower the tension between solid and liquid matter. These compounds can act as emulsifiers, dispersants, detergents, and water infiltration agents. Wetting agents or surfactants reduce surface tension of the liquid and allow it to fully wet and come into contact with optical components (such as the energy guide(s) 122A) and mechanical components (such as other portions of the emitter assembly 129). This reduces or eliminates the accumulation of bubbles and pockets or inclusions of gas within the emitter assembly 129. Nonexclusive examples of chemicals that can be used as wetting agents include, but are not limited to, benzalkonium chloride, benzethonium chloride, cetylpyridinium chloride, Poloxamer 188, Poloxamer 407, Polysorbate 20, Polysorbate 40, and the like. Non-exclusive examples of surfactants can include, but are not limited to, ionic and non-ionic detergents, and sodium stearate. Another suitable surfactant is 4-(5-dodecyl) benzenesulfonate. Other examples can include docusate (dioctyl sodium sulfosuccinate), alkyl ether phosphates, and perfluorooctanesulfonate (PFOS), to name a few.

By using a wetting agent or surfactant, direct liquid contact with the energy guide 122A allows the energy to be more efficiently converted into a plasma. Using the wetting agent or surfactant with the small dimensions of the optical and mechanical components used in the emitter assembly 129 and other parts of the catheter 102, it is less difficult to achieve greater (or complete) wetting. Decreasing the surface tension of the liquid can decrease the difficulty for such small structures to be effectively wetted by the liquid and therefore be nearly or completely immersed. By reducing or eliminating air or other gas bubbles from adhering to the optical and mechanical structure and energy guides 122A, considerable increase in efficiency of the device can occur.

The specific percentage of the wetting agent or surfactant can be varied to suit the design parameters of the catheter system 100 and/or the emitter assembly 129 being used. In one embodiment, the percentage of the wetting agent or surfactant can be less than approximately 50% by volume of the catheter fluid 132. In non-exclusive alternative embodiments, the percentage of the wetting agent or surfactant can be less than approximately 40%, 30%, 20%, 10%, 5%, 2%, 1%, 0.1% or 0.01% by volume of the catheter fluid 132. Still alternatively, the percentage of the wetting agent or surfactant can fall outside of the foregoing ranges.

In some embodiments, the contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine-based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C₅F₁₂).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG−emission maximum=1064 nm) lasers, holmium:YAG (Ho:YAG−emission maximum=2.1 μm) lasers, or erbium:YAG (Er:YAG−emission maximum=2.94 μm) lasers. In some embodiments, the absorptive agents can be water-soluble. In other embodiments, the absorptive agents are not water-soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the one or more energy guides 122A of the energy guide bundle 122 that are in optical communication with the energy source 124. The energy guide(s) 122A can be disposed along the catheter shaft 110 and within the balloon 104.

In some embodiments, each energy guide 122A can be an optical fiber and the energy source 124 can be a laser. The energy source 124 can be in optical communication with the energy guides 122A at the proximal portion 114 of the catheter system 100.

In some embodiments, the catheter shaft 110 can be coupled to multiple energy guides 122A such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; six energy guides 122A can be spaced apart from one another by approximately 60 degrees about the circumference of the guidewire lumen 118 and/or the catheter shaft 110; eight energy guides 122A can be spaced apart from one another by approximately 45 degrees about the circumference of the guidewire lumen 118 and/or the catheter shaft 110; or ten energy guides 122A can be spaced apart from one another by approximately 36 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

In certain embodiments, the guidewire lumen 118 can be substantially annular-shaped and/or cylindrical-shaped and can have a grooved outer surface, with the grooves extending in a generally longitudinal direction along the guidewire lumen 118. In such embodiments, each of the energy guides 122A and/or the emitter(s) 131 of the emitter assembly 129 can be positioned, received and retained within an individual groove formed along and/or into the outer surface of the guidewire lumen 118. Alternatively, the guidewire lumen 118 can be formed without a grooved outer surface, and the position of the energy guides 122A and/or the emitter(s) 131 of the emitter assembly 129 relative to the guidewire lumen 118 can be maintained in another suitable manner.

The catheter system 100, the catheter 102 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100, the catheter 102 and/or the energy guide bundle 122 can include from one energy guide 122A to greater than 30 energy guides 122A. Alternatively, in other embodiments, the catheter system 100, the catheter 102 and/or the energy guide bundle 122 can include greater than 30 energy guides 122A.

The energy guides 122A can have any suitable design that is useful and appropriate for purposes of enabling the generation of plasma and/or pressure waves in the catheter fluid 132 within the balloon interior 146. Thus, the general description of the energy guides 122A as light guides is not intended to be limiting in any manner, except for as set forth in the claims appended hereto. More particularly, although the catheter systems 100 are often described with the energy source 124 as a light source and the one or more energy guides 122A as light guides, the catheter system 100 can alternatively include any suitable energy source 124 and energy guides 122A for purposes of enabling the generation of the desired plasma in the catheter fluid 132 within the balloon interior 146. For example, in one non-exclusive alternative embodiment, the energy source 124 can be configured to provide high-voltage electrical pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the pressure waves in the catheter fluid 132 that are utilized to provide the fracture force onto the vascular lesions 106A at the treatment site 106. Still alternatively, the energy source 124 and/or the energy guides 122A can have another suitable design and/or configuration, be it electrical, acoustic, pneumatic, other mechanical, etc.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P toward the guide distal end 122D, with the guide distal end 122D having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the length of the balloon 104 and/or relative to the length of the guidewire lumen 118 to more effectively and precisely impart pressure waves for purposes of disrupting the vascular lesions 106A at the treatment site 106.

In certain embodiments, the energy guides 122A can include one or more photoacoustic transducers 154, where each photoacoustic transducer 154 can be in optical communication with the energy guide 122A within which it is disposed. In some embodiments, the photoacoustic transducers 154 can be in optical communication with the guide distal end 122D of the energy guide 122A. In such embodiments, the photoacoustic transducers 154 can have a shape that corresponds with and/or conforms to the guide distal end 122D of the energy guide 122A.

The photoacoustic transducer 154 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 154 disposed at the guide distal end 122D of the energy guide 122A can assume the same shape as the guide distal end 122D of the energy guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 154 disposed along one or more side surfaces of the length of the energy guide 122A.

In some embodiments, the energy guides 122A and/or the emitter assembly 129 can further include one or more diverting structures or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting structure can include any structure of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can have any suitable structural configuration that is configured to direct energy in the energy guide 122A toward a side surface that is at or near the guide distal end 122D, where the side surface that is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing energy away from the guide distal end 122D of the energy guide 122A can include, but are not to be limited to, those having a convex surface, a gradient-index (GRIN) lens, and a mirror focus lens. Upon contact with the diverting structure, the energy can be diverted within the energy guide 122A to one or more of the plasma target 133 and the photoacoustic transducer 154 that is in optical communication with a side surface of the energy guide 122A.

When utilized, the plasma target 133 receives energy emitted from the guide distal end 122D of the energy guide 122A to generate plasma in the catheter fluid 132 within the balloon interior 146, which, in turn, causes the creation of plasma bubbles and/or pressure waves that can be directed away from the side surface of the energy guide 122A and toward the balloon wall 130. Additionally, or in the alternative, when utilized, the photoacoustic transducer 154 converts light energy into an acoustic wave that extends away from the side surface of the energy guide 122A.

Additionally, or in the alternative, in certain embodiments, such diverting structures that can be incorporated into the energy guides 122A, can also be incorporated into the design of the emitter assembly 129 and/or the plasma target 133 for purposes of directing and/or concentrating acoustic and mechanical energy toward specific areas of the balloon wall 130 in contact with the vascular lesions 106A at the treatment site 106 to impart pressure onto and induce fractures in such vascular lesions 106A.

The source manifold 136 can be positioned at or near the proximal portion 114 of the catheter system 100. The source manifold 136 can include one or more proximal end openings that can receive the one or more energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132 as needed.

As noted above, in the embodiment illustrated in FIG. 1, the system console 123 includes one or more of the energy source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, and the GUI 127 can be provided within the catheter system 100 without the specific need for the system console 123.

As shown, the system console 123, and the components included therewith, is operatively coupled to the catheter 102, including the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (which can generally include one or more ferrules) that houses a portion, such as at least the guide proximal end 122P, of each of the energy guides 122A. At least a portion of the guide coupling housing 150 is configured to fit and be selectively retained within the console connection aperture 148 to provide the mechanical coupling between the energy guide bundle 122 and the system console 123, as well as helping to provide an optical coupling between the energy source 124 and the energy guides 122A of the energy guide bundle 122.

The energy guide bundle 122 can also include a guide bundler 152 (or “shell”) that brings each of the individual energy guides 122A closer together so that the energy guides 122A and/or the energy guide bundle 122 can be in a more compact form as it extends as part of the catheter 102 into the blood vessel 108 or the heart valve during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122, such as through the use of a multiplexer (not shown), as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104, such as via the plasma target 133 that can be located at or near the guide distal end 122D of the energy guide 122A. In particular, in such embodiments, the energy emitted at the guide distal end 122D of the energy guide 122A is directed toward, contacts, and energizes the plasma target 133 to form the plasma in the catheter fluid 132 within the balloon interior 146. The plasma formation can initiate a pressure wave and can initiate the rapid formation of one or more bubbles that can rapidly expand to a maximum size and then dissipate through a cavitation event that can also launch a pressure wave upon collapse. An exemplary plasma-induced bubble 134 is illustrated in FIG. 1.

The rapid expansion of the plasma-induced bubbles 134 can generate one or more pressure waves within the catheter fluid 132 and thereby impart pressure waves upon the treatment site 106. The pressure waves can transfer mechanical energy through an incompressible catheter fluid 132 to the treatment site 106 to impart a fracture force on the vascular lesions 106A at the treatment site 106. Without wishing to be bound by any particular theory, it is believed that the rapid change in catheter fluid 132 momentum upon the balloon wall 130 of the balloon 104 that is in contact with or positioned near the vascular lesions 106A at the treatment site 106 is transferred to the vascular lesions 106A to induce fractures in the vascular lesions 106A.

In various non-exclusive alternative embodiments, the sub-millisecond pulses of energy from the energy source 124 can be delivered to the treatment site 106 at a pulse frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a pulse frequency that can be greater than 5000 Hz or less than one Hz, or any other suitable range of pulse frequencies.

It is appreciated that although the energy source 124 is typically utilized to provide pulses of energy, the energy source 124 can still be described as providing a single source beam 124A, i.e. a single pulsed source beam.

The energy sources 124 suitable for use can include various types of light sources including lasers and lamps. For example, in certain non-exclusive embodiments, the energy source 124 can be an infrared laser that emits energy in the form of pulses of infrared light. Alternatively, as noted above, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths and energy levels that can be employed to achieve plasma in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 KHz.

In some embodiments, the laser can include a Q-switched thulium: yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium: yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 in the catheter fluid 132.

The catheter system 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In various non-exclusive alternative embodiments, the catheter systems 100 can generate pressure waves having maximum pressures in the range of at least approximately two MPa to 50 MPa, at least approximately two MPa to 30 MPa, or approximately at least 15 MPa to 25 MPa.

The pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to 30 MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, and the handle assembly 128. The power source 125 can have any suitable design for such purposes.

The system controller 126 is electrically coupled to and receives power from the power source 125. The system controller 126 is coupled to and is configured to control operation of each of the energy source 124 and the GUI 127. The system controller 126 can include one or more processors or circuits for purposes of controlling the operation of at least the energy source 124 and the GUI 127. For example, the system controller 126 can control the energy source 124 for generating pulses of energy as desired and/or at any desired firing rate.

The system controller 126 can also be configured to control operation of other components or aspects of the catheter system 100, such as the positioning of the catheter 102 and/or the guide distal end 122D of the energy guides 122A adjacent to the treatment site 106, the inflation of the balloon 104 with the catheter fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. The GUI 127 is electrically connected to the system controller 126. With such design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications and/or desires of the user or operator.

As shown in FIG. 1, the handle assembly 128 can be positioned at or near the proximal portion 114 of the catheter system 100, and/or near the source manifold 136. In this embodiment, the handle assembly 128 is coupled to the balloon 104 and is positioned spaced apart from the balloon 104. Alternatively, the handle assembly 128 can be positioned at another suitable location.

The handle assembly 128 is attached to the catheter shaft 110 and is handled and used by the user or operator to operate, position and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, and the GUI 127.

In some embodiments, the handle assembly 128 can integrate and/or include at least a portion of the system controller 126 within an interior of the handle assembly 128. For example, as shown, in certain embodiments, the handle assembly 128 can include circuitry 156, which is electrically coupled between catheter electronics and the system console 123, and which can form at least a portion of the system controller 126. In some embodiments, the circuitry 156 can transmit such electrical signals or otherwise provide data to the system controller 126.

In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, such as within the system console 123. It is understood that the handle assembly 128 can include fewer or additional components than those specifically illustrated and described herein.

In various embodiments, as noted above, the emitter assembly 129 includes and/or incorporates the at least one emitter 131 that is configured to transmit energy from the energy source 124 into the balloon interior 146 so that plasma and/or pressure waves are generated in the catheter fluid 132 within the balloon interior. Each emitter 131 includes the guide distal end 122D of one of the energy guides 122A and the corresponding plasma target 133 that is positioned near, but typically spaced apart from, the guide distal end 122D. In many embodiments, each emitter 131 further includes the emitter housing 260 that is configured to maintain the desired positioning between the guide distal end 122D of the energy guide 122A and the plasma target 133. As so defined, the emitters 131, and/or the components thereof, are configured to direct and/or concentrate energy generated in the catheter fluid 132 within the balloon interior 146 so as to impart pressure onto and induce fractures in vascular lesions 106A at the treatment site 106.

As noted above, in many embodiments, the emitter 131 further includes the coating 260C that is provided and/or applied on the housing surface 260S of the emitter housing 260. As described herein, the coating 260C that is provided and/or applied on the housing surface 260S is configured to provide enhanced protection and strength for the emitter housing 260, and, thus, enhanced durability for the emitter housing 260 and/or the emitter 131 as a whole.

The emitter housing 260, including the housing surface 260S, and the coating 260C that is provided and/or applied on the housing surface 260S, can be formed from any suitable materials. Various examples of suitable materials for the emitter housing 260, including the housing surface 260S, and the coating 260C are set forth in great detail herein above.

During use of the catheter system 100, the energy guide 122A receives the energy from the energy source 124 and guides the energy from the guide proximal end 122P toward the guide distal end 122D. The energy is then emitted from the guide distal end 122D of the energy guide 122A so that the energy is directed toward and contacts and energizes the corresponding plasma target 133 for purposes of generating the plasma in the catheter fluid 132 within the balloon interior 146. The plasma generation then forms the pressure waves in the catheter fluid 132 that are directed toward the vascular lesions 106A at the treatment site 106 to provide the fracture force onto the vascular lesions 106A at the treatment site 106.

The plasma target 133 can be formed from any suitable material that is configured to generate the desired plasma in the catheter fluid 132 within the balloon interior 146 when the energy is directed from the guide distal end 122D of the energy guide 122A to contact the plasma target 133. More particularly, the plasma target 133 includes a target surface 233S (illustrated in FIG. 2) which is contacted by the energy that is emitted from the guide distal end 122D of the energy guide 122A; and at least the target surface 233S of the plasma target 133 is formed from such material that is configured to generate the desired plasma in the catheter fluid 132 within the balloon interior 146 when the energy contacts the target surface 233S.

In certain non-exclusive embodiments, the plasma target 133 and/or the target surface 233S can be formed, at least in part, from one or more metallics and/or metal alloys having relatively high melting temperatures, such as titanium, stainless steel, tungsten, tantalum, platinum, molybdenum, niobium, iridium, etc. Alternatively, the plasma target 133 and/or the target surface 233S can be formed from at least one of magnesium oxide, beryllium oxide, tungsten carbide, titanium nitride, titanium carbonitride, and titanium carbide. Still alternatively, the plasma target 133 and/or the target surface 233S can be formed from at least one of chemical vapor deposition (CVD) diamond and diamond. In other embodiments, the plasma target 133 and/or the target surface 233S can be formed from a transition metal, an alloy metal, or a ceramic material. Yet alternatively, in some embodiments, the plasma target 133 and/or the target surface 233S can be formed at least partially from a polymer, a polymeric material, and/or a plastic such as polyimide and nylon. Still alternatively, the plasma target 133 and/or the target surface 233S can be formed from any other suitable materials.

Various alternative embodiments of the emitter 131 are illustrated and described in detail herein below within subsequent Figures.

As with all embodiments illustrated and described herein, various structures may be omitted from the figures for clarity and ease of understanding. Additionally, the figures may include certain structures that can be omitted without deviating from the intent and scope of the invention. It is further recognized that the structures included in the various figures shown and described herein are not necessarily drawn to scale for ease of viewing and/or understanding.

FIG. 2 is a simplified schematic side view illustration of a portion of an embodiment of the catheter system 200, including an embodiment of the emitter 231. The design of the catheter system 200 can be varied. In various embodiments, as illustrated in FIG. 2, the catheter system 200 can include a catheter 202 including a catheter shaft 210; a balloon 204 having a balloon wall 230 that defines a balloon interior 246, a balloon proximal end 204P, and a balloon distal end 204D; a catheter fluid 232 that is retained substantially within the balloon interior 246; and the emitter 231, which in certain embodiments can incorporate at least a portion of an energy guide 222A. Alternatively, in other embodiments, the catheter system 200 can include more components or fewer components than what is specifically illustrated and described herein. For example, certain components that were illustrated in FIG. 1, such as the guidewire 112, the guidewire lumen 118, the source manifold 136, the fluid pump 138, the energy source 124, the power source 125, the system controller 126, the GUI 127, and the handle assembly 128, are not specifically illustrated in FIG. 2 for purposes of clarity, but would likely be included in any embodiment of the catheter system 200.

The design and function of the catheter shaft 210, the balloon 204, and the catheter fluid 232 are substantially similar to what was illustrated and described herein above. Accordingly, a detailed description of such components will not be repeated.

The balloon 204 is again selectively movable between a deflated state suitable for advancing the catheter 202 through a patient's vasculature, and an inflated state suitable for anchoring the catheter 202 in position relative to the treatment site 106 (illustrated in FIG. 1). In some embodiments, the balloon proximal end 204P can be coupled to the catheter shaft 210, and the balloon distal end 204D can be coupled to the guidewire lumen 118 (illustrated in FIG. 1). The balloon 204 can again be inflated with the catheter fluid 232, such as from the fluid pump 138 (illustrated in FIG. 1), that is directed into the balloon interior 246 of the balloon 204 via the inflation conduit 140 (illustrated in FIG. 1).

In various embodiments, the emitter 231 is configured to direct and/or concentrate energy generated in the catheter fluid 232 within the balloon interior 246 to impart pressure onto and induce fractures in vascular lesions 106A (illustrated in FIG. 1) at the treatment site 106. More particularly, the emitter 231 is configured to direct and concentrate acoustic and/or mechanical energy toward specific areas of the balloon wall 230 that are in contact with the vascular lesions 106A at the treatment site 106 to enhance the delivery of such energy to the treatment site 106. As illustrated in this embodiment, at least some of the components of the emitter 231 are positioned within the balloon interior 246.

The design of the emitter 231 can be varied. As shown in FIG. 2, in certain embodiments, the emitter 231 includes at least a portion of the energy guide 222A, such as at least the guide distal end 222D of the energy guide 222A, a plasma target 233, and an emitter housing 260 that is coupled to and/or secured to the energy guide 222A and the plasma target 233.

As illustrated in this embodiment, the emitter 231 includes the guide distal end 222D of the energy guide 222A and the corresponding plasma target 233 that is positioned near, but typically spaced apart from, the guide distal end 222D. During use of the catheter system 200, the energy guide 222A emits energy 243 (such as light energy in certain non-exclusive embodiments, illustrated as a dashed arrow in FIG. 2) from the guide distal end 222D of the energy guide 222A toward the plasma target 233. In certain embodiments, the energy guide 222A includes a distal region 247 having a longitudinal axis 249, and the direction the energy 243 is emitted from the energy guide 222A is substantially along the longitudinal axis 249 of the distal region 247 of the energy guide 222A.

As shown, the plasma target 233 is spaced apart from the guide distal end 222D of the energy guide 222A by a target gap distance 245. The target gap distance 245 can vary. For example, in various embodiments, the target gap distance 245 can be at least 1 μm, at least 10 μm, at least 100 μm, at least 1 mm, at least 2 mm, at least 3 mm, at least 5 mm, or at least 1 cm. The target gap distance 245 can vary depending upon the size, shape and/or angle of the plasma target 233 relative to the energy 243 emitted by the energy guide 222A, the type of material used to form the plasma target 233, the quantity and/or duration of the energy 243 being emitted from the energy guide 222A, the type of catheter fluid 232 used in the balloon 204, etc.

With this design, the energy 243 being emitted from the guide distal end 222D of the energy guide 222A and impinging on or contacting the plasma target 233 generates a plasma bubble 134 (illustrated in FIG. 1), which creates an outwardly emanating pressure wave (not shown) throughout the catheter fluid 232 that impacts the balloon 204. The impact to the balloon 204 causes the balloon 204 to forcefully disrupt and/or fracture the vascular lesion 106A (illustrated in FIG. 1), such as a calcified vascular lesion, at the treatment site 106 (illustrated in FIG. 1). In other words, the associated rapid formation of the plasma bubble 134 and resulting localized catheter fluid 232 velocity within the balloon 204 transfers mechanical energy though the incompressible catheter fluid 232 to impart a fracture force on the treatment site 106. The rapid change in momentum of the catheter fluid 232 upon hitting the balloon wall 230 is known as hydraulic shock, or water hammer. The change in momentum of the catheter fluid 232 is transferred as a fracture force to the vascular lesion 106A which is opposed to the balloon wall 230.

By positioning the plasma target 233 away from the guide distal end 222D of the energy guide 222A, damage to the energy guide 222A from the plasma bubble 134 is less likely to occur than if the plasma bubble 134 was generated at or more proximate the guide distal end 222D of the energy guide 222A. Stated another way, the presence of the plasma target 233, and positioning the plasma target 233 away from the guide distal end 222D of the energy guide 222A, causes the plasma bubble 134 to in turn be generated away from the guide distal end 222D of the energy guide 222A, reducing the likelihood of damage to the energy guide 222A.

The emitter housing 260 is configured to maintain the desired positioning between the guide distal end 222D of the energy guide 222A and the plasma target 233 (such as the desired target gap distance 245), and, in conjunction with the guide distal end 222D and the corresponding plasma target 233, to direct and/or concentrate energy generated in the catheter fluid 232 within the balloon interior 246 so as to impart pressure onto and induce fractures in vascular lesions 106A at the treatment site 106 within or adjacent to a vessel wall 108A (illustrated in FIG. 1) of a blood vessel 108 (illustrated in FIG. 1), or within or adjacent to a heart valve. More particularly, by effectively maintaining the desired positioning between the guide distal end 222D of the energy guide 222A and the plasma target 233, and with the particular design features that may be incorporated into the emitter 231, the emitter 231 is configured to concentrate and direct acoustic and/or mechanical energy toward specific areas of the balloon wall 230 in contact with the vascular lesions 106A at the treatment site 106 to enhance the delivery of such energy to the treatment site 106. Thus, the emitter 231 is able to effectively improve the efficacy of the catheter system 200.

It is appreciated that, in some embodiments, a separate emitter 231 can be included with and/or incorporated into each individual energy guide 222A. Alternatively, in other embodiments, a single emitter 131 can be configured to operate in conjunction with more than one energy guide 222A. Still alternatively, each energy guide 222A need not have an emitter 131 incorporated therein or associated therewith.

In some embodiments, as illustrated, the emitter housing 260 can include one or more of (i) a first housing section 262 that is coupled and/or secured to the energy guide 222A, such as at or near the guide distal end 222D of the energy guide 222A, (ii) a second housing section 264 that is coupled and/or secured to the plasma target 233, and (iii) a connector section 266 that is coupled to, integrally formed with, and/or extends between the first housing section 262 and the second housing section 264. In certain embodiments, the emitter housing 260 can be formed as a unitary structure that includes each of the first housing section 262, the second housing section 264 and the connector section 266. In other embodiments, the first housing section 262, the second housing section 264 and/or the connector section 266 of the emitter housing 260 can be formed as separate components that are secured to one another. Alternatively, the emitter housing 260 can include more components or fewer components than what is specifically illustrated in FIG. 2.

As shown, the first housing section 262 of the emitter housing 260 is configured to be secured to and substantially encircle at least a portion of the energy guide 222A, such as at or near the guide distal end 222D of the energy guide 222A. In one embodiment, the first housing section 262 of the emitter housing 260 is substantially annular-shaped and/or cylindrical-shaped, and includes a guide aperture 262A through and/or into which the energy guide 222A can be positioned. Alternatively, the first housing section 262 can have another suitable shape.

As utilized herein, the description of the first housing section 262 as substantially encircling at least a portion of the energy guide 222A and/or being substantially annular-shaped and/or cylindrical-shaped is intended to signify that the first housing section 262 encircles at least approximately 90% to 95% of such portion of the energy guide 222A, but can further include a small housing gap (not shown) that extends fully along a length of the first housing section 262 and that allows for slight expansion or contraction of the first housing section 262 due to changes in environmental conditions in which the catheter system 200 is being used. The housing gap allows for such potential expansion or contraction of the first housing section 262 without adversely impacting the structure of the guide distal end 222D of the energy guide 222A about which the first housing section 262 is positioned.

The first housing section 262 can be secured to a portion of the energy guide 222A, such as at or near the guide distal end 222D, in any suitable manner. For example, the first housing section 262 can be secured to a portion of the energy guide 222A with any suitable type of adhesive material 263. Alternatively, the first housing section 262 can be secured to a portion of the energy guide 222A in another suitable manner.

Somewhat similarly, as shown, the second housing section 264 of the emitter housing 260 is configured to be secured to and substantially encircle the plasma target 233. In one embodiment, the second housing section 264 of the emitter housing 260 is substantially annular-shaped and/or cylindrical-shaped, and includes a target aperture 264A through and/or into which the plasma target 233 can be positioned. Alternatively, the second housing section 264 can have another suitable shape.

As utilized herein, the description of the second housing section 264 as substantially encircling the plasma target 233 and/or being substantially annular-shaped and/or cylindrical-shaped is intended to signify that the second housing section 264 encircles at least approximately 90% to 95% of the plasma target 233, but can further include a small housing gap (not shown) that extends fully along a length of the second housing section 264 and that allows for slight expansion or contraction of the second housing section 264 due to changes in environmental conditions in which the catheter system 200 is being used. The housing gap allows for such potential expansion or contraction of the second housing section 264 without adversely impacting the structure of the plasma target 233 about which the second housing section 264 is positioned.

The second housing section 264 can be secured to the plasma target 233 in any suitable manner. For example, the second housing section 264 can be secured to the plasma target 233 with any suitable type of adhesive material 263. Alternatively, the second housing section 264 can be secured to the plasma target 233 in another suitable manner.

The connector section 266 of the emitter housing 260, as noted, is coupled to, integrally formed with, and/or extends between the first housing section 262 and the second housing section 264. In some embodiments, the connector section 266 can be partially annular-shaped and/or cylindrical-shaped, with a section opening 272 that extends fully along a length of the connector section 266 to help define the less than complete annular and/or cylindrical shape of the connector section 266, and that is configured such that the plasma energy formed in the catheter fluid 232 within the balloon interior 246 is directed and/or concentrated through the section opening 272 and toward the vascular lesions 106A formed at the treatment site 106. The size and orientation of the section opening 272 can be varied depending on the size and position of the vascular lesions 106A being treated with the catheter system 200. In some non-exclusive alternative embodiments, the section opening 272 can be less than approximately 10%, 15%, 20%, 25%, 30%, 35%, 40%, 45%, 50%, 55%, 60%, 65%, 70%, 75%, 80%, 85% or 90% of what would otherwise form a complete annular and/or cylindrical shape for the connector section 266. Stated in another manner, the connector section 266 can be formed as at least approximately 90%, 85%, 80%, 75%, 70%, 65%, 60%, 55%, 50%, 45%, 40%, 35%, 30%, 25%, 20%, 15% or 10% of a complete annular-shaped and/or cylinder-shaped body.

As noted above, the emitter housing 260 can be thin, long and narrow, and can be subject to certain manufacturing irregularities that can weaken the overall structural integrity of the emitter housing 260. More specifically, during the manufacturing process and/or during repeated use of the catheter system 200 (and thus the emitter housing 260), the emitter housing 260 can include and/or develop small surface imperfections, such as cracks, crevices, surface roughness and/or other surface irregularities, that can adversely impact the structural strength of the emitter housing 260. It is appreciated that, due to its smaller size relative to a complete annular-shaped and/or cylinder-shaped body in order to define the section opening 272, the connector section 266 can be the most susceptible to potential breaking or failure as a result of any surface imperfections that can adversely impact the structural strength of the emitter housing 260.

In various embodiments, the emitter housing 260 can be formed from long, narrow tubing (such as a hypotube) and from any suitable materials. For example, in certain non-exclusive embodiments, the emitter housing 260 can be formed from a hypotube including one or more metals such as titanium, stainless steel, tungsten, tantalum, platinum, molybdenum, niobium, iridium, etc. Alternatively, the emitter housing 260 may be formed from a hypotube including plastics such as polyimide and nylon. Still alternatively, the emitter housing 260 may be injection molded, and over molding processing can be used to secure either the energy guide 222A and/or the plasma target 233 into place. Yet alternatively, the emitter housing 260 can be formed in another suitable manner and/or from other suitable materials. For example, in certain alternative embodiments, the features on the emitter housing 260 can be done by laser cutting, milling, or swiss screw machining processes of a raw hypotube.

As also shown in FIG. 2, and as noted above, the emitter housing 260 can further include a coating 260C that is provided and/or applied on a housing surface 260S of the emitter housing 260. As noted above, the coating 260C is configured to provide enhanced protection and strength for the emitter housing 260, and, thus, enhanced durability for the emitter housing 260 and/or the emitter 231 as a whole.

As further noted above, the coating 260C can be formed from any suitable materials for purposes of filling in any cracks or crevices that may have formed on or into the housing surface 260S, smoothing out any rough surfaces within the housing surface 260S, and/or reducing a depth of any surface irregularities that may have been formed into the housing surface 260S. In certain non-exclusive embodiments, the coating 260C can be formed from one or more of CVD diamond material, dip or spray-coated adhesives and/or polymers, gold plating, or any other suitable materials that can also be used to strengthen the housing surface 260S of the emitter housing 260 and/or the emitter 231 as a whole.

The coating 260C can be provided and/or applied on the housing surface 260S to have any desired thickness 261. In certain embodiments, the coating 260C can have a thickness 261 of between a few microns and several millimeters. For example, in some non-exclusive embodiments, the coating 260C can have a thickness 261 of between approximately one micron and fifteen millimeters, between approximately two microns and ten millimeters, between approximately three microns and eight millimeters, between approximately three microns and six millimeters, or within another suitable range. Alternatively, the coating 260C can have a thickness 261 of greater than approximately fifteen millimeters or less than approximately one micron.

With the noted design of the emitter housing 260, a desired relative positioning can be effectively maintained between the guide distal end 222D of the energy guide 222A and the plasma target 233. During use of the catheter system 200, energy can be transmitted through the energy guide 222A and can be directed through the guide distal end 222D and toward the plasma target 233 such that plasma can be generated in the catheter fluid 232 within the balloon interior 246 of the balloon 202. The guide distal end 222D can have any suitable shape such that the energy transmitted through the energy guide 222A can be effectively and accurately directed through the guide distal end 222D and toward the plasma target 233. In one embodiment, the guide distal end 222D can have a flat, cleaved end through which the energy is directed toward the plasma target 233. Alternatively, the guide distal end 222D can be generally semi-spherical, ball-shaped, conical, wedge-shaped, pyramidal, angled, or can be another suitable shape.

In some embodiments, as shown in FIG. 2, the plasma target 233 can include a proximal end 233P that is angled or otherwise configured to more effectively direct and/or concentrate the energy in the form of the plasma and/or pressure waves that have been generated in the catheter fluid 232 through the section opening 272 in the connector section 266 of the emitter housing 260 and toward the balloon wall 230 positioned adjacent to the vascular lesions 106A at the treatment site 106. It is appreciated that the proximal end 233P of the plasma target 233 can be configured at any suitable angle so as to effectively direct and/or concentrate the plasma energy and/or the pressure waves as desired. For example, in some embodiments, the proximal end 233P of the plasma target 233 can be angled at between approximately 5 degrees and 45 degrees relative to a flat, perpendicular configuration. Alternatively, the proximal end 233P of the plasma target 233 can be angled at less than 5 degrees or greater than 45 degrees relative to a flat, perpendicular configuration in order to direct energy in the form of the plasma and/or the pressure waves that have been generated in the catheter fluid 232 toward the balloon wall 230 positioned adjacent to the treatment site 106.

As with the previous embodiments, the plasma target 233 can have a target surface 233S, and the plasma target 233 and/or the target surface 233S can be formed from any suitable materials that are configured to generate the desired plasma in the catheter fluid 232 within the balloon interior 246 when the energy is directed from the guide distal end 222D of the energy guide 222A to impinge on or contact the target surface 233S of the plasma target 233. For example, in certain non-exclusive embodiments, the plasma target 233 and/or the target surface 233S can again be formed from one or more metals or metal alloys such as titanium, stainless steel, tungsten, tantalum, platinum, molybdenum, niobium, iridium, etc. Alternatively, the plasma target 233 and/or the target surface 233S can be formed from at least one of magnesium oxide, beryllium oxide, tungsten carbide, titanium nitride, titanium carbonitride, and titanium carbide. Still alternatively, the plasma target 233 and/or the target surface 233S can be formed from at least one of chemical vapor deposition (CVD) diamond and diamond. In other embodiments, the plasma target 233 and/or the target surface 233S can be formed from a transition metal, an alloy metal, or a ceramic material. Yet alternatively, in some embodiments, the plasma target 233 and/or the target surface 233S can be formed at least partially from a polymer, a polymeric material, and/or a plastic such as polyimide and nylon. Still alternatively, the plasma target 233 and/or the target surface 233S can be formed from any other suitable materials. It is appreciated that in different embodiments, the plasma target 233 and/or the target surface 233S can be formed from the same materials as the emitter housing 260 or different materials from the emitter housing 260.

The target surface 233S at the proximal end 233P of the plasma target 233 can also have any suitable geometry, shape or configuration. For example, in some non-exclusive alternative embodiments, the target surface 233S at the proximal end 233P of the plasma target 233 can have a geometry, shape and/or configuration including, but not limited to a somewhat conical configuration, a somewhat pyramidal configuration, a somewhat convex or dome-shaped configuration, a somewhat concave configuration, a spiral projection that extends outwardly from a side portion of the plasma target, a somewhat spring-like or coiled configuration, a toroidal or frustoconical configuration, or a beveled configuration. The target surface 233S at the proximal end 233P of the plasma target 233 can also have any suitable cross-sectional shape. For example, in certain non-exclusive embodiments, the target surface 233S at the proximal end 233P of the plasma target 233 can have a cross-sectional shape that is substantially circular, vertical oval or elliptical, square, diamond, trapezoidal, parallelogram, hexagonal, horizonal oval or elliptical, pentagonal, octagonal, vertical rectangular, or horizonal rectangular. In some embodiments, the target surface 233S at the proximal end 233P of the plasma target 233 can also include one or more surface features, such as dimples, depressions or indentations that extend into the target surface 233S, or projections that extend outwardly from the target surface 233S. In one embodiment, the surface features can include the same or other materials that are added to the target surface 233S. The specific sizes and/or shape(s) of the surface features can be varied.

It is appreciated that during use of the catheter system 200, the catheter fluid 232 that is utilized to inflate the balloon 204 is also allowed to enter into the area of the connector section 266 of the emitter housing 260 through the section opening 272. Subsequently, the pulsed energy that is directed through the energy guide 222A and toward the plasma target 233 generates a plasma-induced bubble 134 (illustrated in FIG. 1) in the catheter fluid 232 near and/or adjacent to the target surface 233S at the proximal end 233P and/or in the general area of the connector section 266 of the emitter housing 260. As the bubble 134 expands, it is directed and/or focused by the proximal end 233P of the plasma target 233 through the section opening 272 of the connector section 266 and toward the balloon wall 230 positioned adjacent to the vascular lesions 106A at the treatment site 106.

FIG. 3 is a simplified schematic perspective view illustration of a portion of another embodiment of the catheter system 300, more particularly illustrating another embodiment of the emitter 331. As shown in FIG. 3, the emitter 331 is somewhat similar in design, positioning and function to the embodiment illustrated and described in detail in relation to FIG. 2. In this embodiment, the emitter 331 again includes at least part of an energy guide 322A, such as at least the guide distal end 322D of the energy guide 322A, a plasma target 333, and an emitter housing 360. The emitter housing 360 again also includes (i) a first housing section 362, including a guide aperture 362A, that is configured to at least substantially encircle a portion of the energy guide 322A, such as at or near a guide distal end 322D of the energy guide 322A; (ii) a second housing section 364; and (iii) a connector section 366, again including a section opening 372, that is coupled to, integrally formed with and/or extends between the first housing section 362 and the second housing section 364. In this embodiment, the emitter 331 is again configured to effectively direct and/or concentrate energy generated in the catheter fluid 232 (illustrated in FIG. 2) that is retained within the balloon 204 (illustrated in FIG. 2) so as to impart pressure onto and induce fractures in the vascular lesions 106A (illustrated in FIG. 1) at the treatment site 106 (illustrated in FIG. 1).

However, as shown in the embodiment illustrated in FIG. 3, the plasma target 333 is integrally formed with the second housing section 364 of the emitter housing 360, rather than being positioned and/or secured substantially within the second housing section as in the previous embodiment.

With such design, the emitter housing 360 can be fabricated by machining a single piece of rod instead of making it out of a hypotube, as is typically used for the embodiment of the emitter housing 260 illustrated in FIG. 2. Machining the emitter housing 360 can be achieved through the use of any suitable machining processes. For example, in certain non-exclusive implementations, machining of the emitter housing 360 can be achieved by electrical discharge machining, or micro machining using milling or swiss screw machining techniques.

It is appreciated that this design provides certain key advantages as compared to the hypotube design utilized in the embodiment of FIG. 2, some of which are specifically noted below. First, this integrated design where the emitter housing 360 is formed from a single piece of rod allows for the guide aperture 362A that is formed into the first housing section 362 and is configured to receive and retain the portion of the energy guide 322A to be offset from a central axis 374 of the emitter housing 360, therefore allowing the connector section 366 to be thicker in dimension. With the connector section 366 being thicker in dimension, the connector section 366 will also be somewhat less susceptible to damage or failure from surface imperfections that can adversely impact the structural strength of the emitter housing 360. Second, this integrated design maximizes the cross-sectional area of the plasma target 333 since it is made out of the same material as the second housing section 364. It is desirable to maximize the cross-section of the plasma target 333 to reduce the need for precise alignment of the guide distal end 322D of the energy guide 322A relative to the plasma target 333. Third, radii 376 can be cut into the emitter housing 360 to reduce the number of sharp edges on the emitter housing 360, to inhibit potential damage to the balloon 204. Fourth, compared to the hypotube design, there is no need to bond a plasma target into the emitter housing 360 since the plasma target 333 is already integrated within the second housing section 364 of the emitter housing 360.

Despite the emitter housing 360 being formed from a single piece of rod instead of from a hypotube, it is appreciated that the emitter housing 360 can still be formed from any suitable materials, such as described in detail herein above.

As with the previous embodiments, the plasma target 333 can have a target surface 333S, and the plasma target 333 and/or the target surface 333S of the plasma target 333 can be formed from any suitable materials, such as described in detail herein above. The particular materials of the plasma target 333 and/or the target surface 333S are again configured to generate the desired plasma in the catheter fluid 232 within the balloon interior 246 (illustrated in FIG. 2) when the energy is directed from the guide distal end 322D of the energy guide 322A to impinge on or contact the target surface 333S of the plasma target 333.

Additionally, as with previous embodiments, the emitter housing 360 can again include a coating 360C that is provided and/or applied on a housing surface 360S of the emitter housing 360 to provide enhanced protection and strength for the emitter housing 360, and, thus, enhanced durability for the emitter housing 360 and/or the emitter 331 as a whole. However, in this embodiment, since the plasma target 333 is integrally formed with the emitter housing 360, with the second housing section 364 of the emitter housing 360 in particular, it is appreciated that the coating 360C can also be applied onto a surface of the plasma target 333.

The coating 360C can again be formed from any suitable materials such as described in detail herein above. The coating 360C can also again be provided to have any suitable thickness such as described in detail herein above.

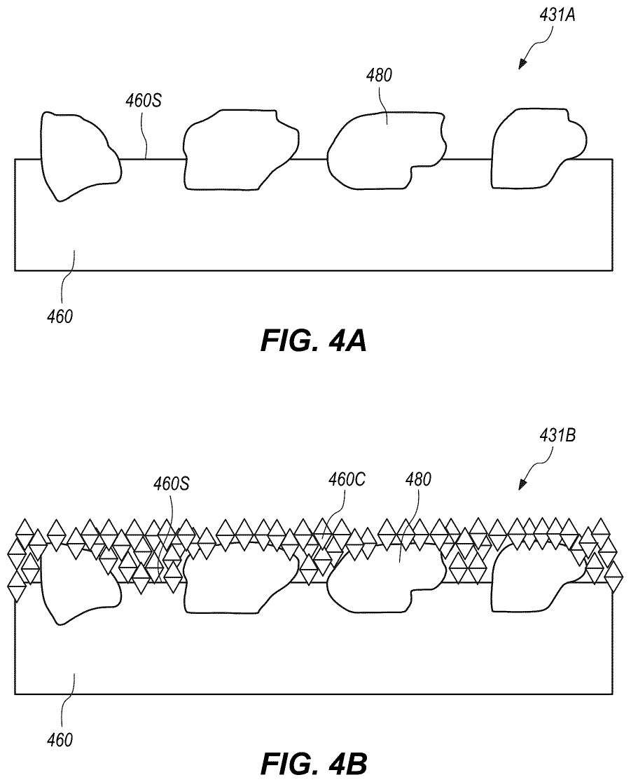

FIG. 4A is a simplified schematic side view illustration of a portion of an embodiment of the emitter 431A. More particularly, FIG. 4A is a simplified schematic side view illustration of a portion of an emitter housing 460 having a housing surface 460S, but without a coating being shown as provided on the housing surface 460S of the emitter housing 460. As illustrated, in certain embodiments, the housing surface 460S can be formed at least partially from and/or can include a plurality of surface grains 480, such as exposed tungsten grains in one non-exclusive embodiment. During repeated use of the catheter system 100 (illustrated in FIG. 1) and due to any potential manufacturing imperfections of the small diameter emitter housing 460, the housing surface 460S can become warn with various imperfections, including crevices, rough surfaces, microcracks, surface irregularities, and the like, which can lead the emitter 431A and/or the emitter housing 460 to fracture when acoustic energy is created.

FIG. 4B is a simplified schematic side view illustration of a portion of another embodiment of the emitter 431B. More particularly, FIG. 4B is a simplified schematic side view illustration of the emitter housing 460 of FIG. 4A, but further including a coating 460C that has been provided on the housing surface 460S of the emitter housing 460. As illustrated, the housing surface 460S can again be formed at least partially from and/or can include the plurality of surface grains 480, such as exposed tungsten grains in one non-exclusive embodiment. However, as shown in FIG. 4B, the coating 460C is provided on the housing surface 460S of the emitter housing 460 in order to fill and/or reduce a depth of any crevices, rough surfaces, microcracks, surface irregularities, and the like, which may have been formed into the housing surface 460S and/or the surface grains 480 during repeated use of the catheter system 100 (illustrated in FIG. 1), and/or due to any potential manufacturing imperfections of the small diameter emitter housing 460. In certain embodiments, as noted herein, the coating 460C can be formed at least partially from a diamond material, such as a CVD diamond material in one non-exclusive embodiment, and can be used to fill and/or reduce the depth of any such crevices, rough surfaces, microcracks, surface irregularities, and the like. Alternatively, the coating 460C can be formed from other suitable materials such as described in detail herein above. The coating 460C can also again be provided to have any suitable thickness such as described in detail herein above.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

It is recognized that the figures shown and described are not necessarily drawn to scale, and that they are provided for ease of reference and understanding, and for relative positioning of the structures.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter system, the emitter, and/or the emitter housing have been illustrated and described herein, one or more features of any one embodiment can be combined with one or more features of one or more of the other embodiments, provided that such combination satisfies the intent of the present invention.

While a number of exemplary aspects and embodiments of the catheter system, the emitter, and/or the emitter housing have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

What is claimed is:

1. A catheter system for treating a treatment site within or adjacent to a vessel wall of a blood vessel, or within or adjacent to a heart valve, within a body of a patient, the catheter system comprising:
an energy source that generates energy;
an energy guide that receives the energy from the energy source, the energy guide having a guide distal end; and
an emitter including (i) the guide distal end of the energy guide, (ii) a corresponding plasma target that is spaced apart from the guide distal end, the energy guide emitting the energy in a direction away from the guide distal end and toward the plasma target so that a plasma is generated at the plasma target upon receiving the energy from the energy guide, and (iii) an emitter housing that is secured to the energy guide and is one of secured to and integrally formed with the plasma target so as to maintain a relative position between the guide distal end of the energy guide and the plasma target, the emitter housing including a housing surface, and with a coating being provided on the housing surface;
wherein the housing surface is formed at least in part from a first material; and
wherein the coating is formed at least in part from a second material that is different than the first material.

2. The catheter system of claim 1 wherein the housing surface is formed at least in part from one or more of titanium, stainless steel, tungsten, tantalum, platinum, molybdenum, niobium, and iridium.

3. The catheter system of claim 2 wherein the housing surface is formed at least in part from tungsten.

4. The catheter system of claim 1 wherein the housing surface is formed at least in part from one or more of a polymer, a polymeric material, a plastic, and nylon.

5. The catheter system of claim 1 wherein the plasma target is formed from the same materials as the housing surface.

6. The catheter system of claim 1 wherein the plasma target is formed from different materials than the housing surface.

7. The catheter system of claim 1 wherein the coating that is provided on the housing surface is formed at least in part from one or more of CVD diamond, diamond, an adhesive, a spray-coated adhesive, and a gold plating.

8. The catheter system of claim 7 wherein the coating that is provided on the housing surface is formed at least in part from CVD diamond.

9. The catheter system of claim 1 wherein the coating that is provided on the housing surface has a thickness of between approximately one micron and fifteen millimeters.

10. The catheter system of claim 1 wherein the coating that is provided on the housing surface has a thickness of between approximately three microns and eight millimeters.

11. The catheter system of claim 1 wherein the emitter housing includes (i) a first housing section that is secured to the energy guide at or near the guide distal end, (ii) a second housing section that is one of secured to and integrally formed with the plasma target, and (iii) a connector section that is coupled to and extends between the first housing section and the second housing section.

12. The catheter system of claim 11 wherein the first housing section includes a guide aperture, at least a portion of the energy guide being secured within the guide aperture.

13. The catheter system of claim 11 wherein the second housing section includes a target aperture, at least a portion of the plasma target being secured within the target aperture.

14. The catheter system of claim 11 wherein the second housing section is integrally formed with the plasma target.

15. The catheter system of claim 11 wherein the plasma generated at the plasma target is in the form of a plasma bubble; and wherein the connector section includes a section opening, the plasma target being configured to direct the energy from the plasma bubble through the section opening and toward the treatment site.

16. The catheter system of claim 15 wherein the plasma target has a proximal end that is angled so the energy from the plasma bubble is directed through the section opening and toward the treatment site.

17. The catheter system of claim 1 wherein the plasma target is spaced apart from the guide distal end by a target gap distance that is greater than 1 μm.

18. The catheter system of claim 1 further comprising an inflatable balloon that encircles the guide distal end of the energy guide, the plasma target being positioned within the inflatable balloon.

19. The catheter system of claim 1 wherein the energy guide includes a distal region having a longitudinal axis, and wherein the direction the energy is emitted from the energy guide and toward the plasma target is substantially along the longitudinal axis of the distal region.

20. The catheter system of claim 1 wherein the energy source is a laser and the energy guide is an optical fiber.
